(19) United States
(12) Patent Application Publication
(10) Pub. No.: US 2005/0277657 A1
(43) Pub. Date: Dec. 15, 2005

(54) Method of protection of biologically active essential oils and pharmaceutical composition for weight control and enhance fat burning in adults, adolescents and children

(76) Inventor: Dusan Ninkov, San Diego, CA (US)

Correspondence Address: DUSAN NINKOV, 11839 Caminito Corriente, San Diego, CA, 92128, US

(21) Appl. No.: 11/143,192

(22) Filed: Jun. 2, 2005

Related U.S. Application Data
(60) Provisional application No. 60/579,630, filed on Jun. 15, 2004.

(51) Int. Cl. A61K 31/522; A61K 31/353

(52) U.S. Cl. 514/263.31; 514/456

(57) ABSTRACT

The subject of this invention is overweight and obesity. The invention relates to use of several natural sources of essential oils such as Cinnamon, Ginger and Turmeric oils. Additional compounds are Citric acid as a fat dissolver and Cocoa extract as an energizer. Especially developed technology is necessary to be used for manufacturing this product. In the first phase powders are separately mixed with oils that are added to inferior carriers (cellulose) to prevent reaction. At the end, Citric acid and Cocoa powder is added as an energizer. The final product Slimtrax® will be in a tablet form and will have appetite suppressant, fat burner and energizing effect. Product should be taken 15 minutes before the main meal with a large (8oz.) glass of water.

### REFERENCES CITED

U.S. Patent Documents:

U.S. Pat. No. 6,723,730 Apr. 20, 2004, Bakthavatchalam, et al.
U.S. Pat. No. 6,518,315 Feb. 11, 2003, Roufogalis, B D. et al.
20020082670 Jun. 27, 2002, Utley, D S. et al.
U.S. Pat. No. 6,214,831 B1 Apr. 10, 2001, Yokoo, Y. et al.
U.S. Pat. No. 5,273,754 Dec. 28, 1993, Mann.

### OTHER PUBLICATIONS

Dedov V N, Tran V H, Duke C C, Connor M, Christie M J, Mandadi S, Roufogalis B D. Gingerols: a novel class of vanilloid receptor (VR1) agonists. Br J Pharmacol. 137(6):793-8, 2002.
Falchi M, Ferrara F, Dib B., Gharib C.; Intracerebroventricular capsaicin and food intake in the rat. Drugs Exptl Clin Res 27(2) 61-67, 2001.
Lejeune M. P. G. M.[1]; Kovacs E. M. R.[1]; Westerterp-Plantenga M. S. Effect of capsaicin on substrate oxidation and weight maintenance after modest bodyweight loss in human subjects. Brit J. Nutr. 90:3, 651-660, 2003.
Martinet, A. Hosterman, K., Schutz, Y. Thermogenic effects of commercially available plant preparations aimed at treating human obesity. Phytomedicine 6(4):231-238, 1999.
Sherman, B. Jolly no more, new research is piecing together the answers to fat's ponderous puzzle. Acumen, II(1):66-67, 2003.
Szallasi, A. Blumberg P M. Vanilloid (Capsaicin) Receptors and Mechanisms, Pharmacol Rev. 51(2): 159-212, 1999.
Yang, B. H. Piao, Z. G. Kim, Y.-B. Lee, C.-H. Lee, J. K. Park, K. Kim, J. S. Oh, S. B. Activation of vanilloid receptor 1 (VR1) by eugenol. J. Dent Res. 82(10):781-785, 2003.
Yoshioka M, St-Pierre S, Drapeau V, Dionne I, Doucet E, Suzuki M, Tremblay A. Effects of red pepper on appetite and energy intake. Br J Nutr. 82(2):115-23, 1999.

### FIELD OF INVENTION

The present invention relates to a method for blocking irreversible inactivation of vanilloids, chemical constituents of essential oils that contain a vanillyl (4-Hydroxy-3-methoxybenzyl) moiety.

Essential oils are incorporated in a pharmaceutically acceptable diluent or carrier mixture that reduces their pungency and reactivity, protecting their effectiveness, and allowing a sustained and sequential release of vanilloids for a long period of time.

The method consists of incorporation of individual essential oils into olive oil followed by incorporation of olive oil-essential oil droplets into untreated fumed silica. The method can be applied to therapeutically effective amounts of essential oils such as cinnamon leaf, ginger, turmeric, thyme and Winter savory oils containing eugenol, carvacrol, gingerol and curcumin among others.

Specifically, the present invention relates to a pharmaceutical composition in tablet form for adults and adolescents that comprises of therapeutically effective amounts of cinnamon leaf oil, ginger extract, oleoresin or oil, turmeric extract oil or oleoresin and combinations of these oils with cocoa extract, citric acid, citrus essential oils and a white kidney bean protein fraction with alpha amylase inhibitory activity.

Specifically, the present invention relates to a pharmaceutical composition in syrup form for children that comprises of therapeutically effective amounts of cinnamon leaf oil, turmeric extract oil or oleoresin and combinations of these oils with cocoa powder, citric acid, citrus essential oils and a white kidney bean protein fraction with alpha amylase inhibitory activity.

Said tablet and syrup are administered to individuals before a meal, to curve their hunger, boost fat metabolism, shift their thermoregulatory balance and reduce sugar intake, simultaneously promoting calorie reduction and weight loss.

### OBJECT AND DESCRIPTION OF THE INVENTION

The new statistics revealed in 2004 by the Centers for Disease Control and Prevention (CDC) of the U.S. government reveal than more than 64 percent of adults in the U.S. are overweight or obese. Of these, 64.5 percent are overweight (BMI of 25 or higher) and 30.5 percent are obese (BMI of 30 or higher).

Keep a healthy weight for is important for both cosmetic and medical reasons. Excess body weight increases risk for developing diabetes, high blood pressure, high cholesterol, heart disease, stroke, some cancers, arthritis, and other chronic conditions. The obesity/disease link is more evident when looking at rates of adult onset diabetes. Approximately 17 million Americans adults have diabetes type II.

In regards to children and adolescent population, the American Obesity Association has suggested this is a serious issue that treats as many as 20-22% of adolescents (ages 12-19) and 19% of children (ages 6 to 11). This trend is growing at a very alarming rate and in some countries is spiraling out of control.

A number of mortality cases attributed to children obesity have been reported. A recent case in Britain of a child aged three that died from heart failure after becoming grossly overweight and four more cases of children who needed help to breath at night because they were so overweight they were chocking on their own fat.

Although the rate of mortality caused by children and adolescents is still low, children and adolescent obesity has many health and social consequences that often continue into adulthood. Pediatricians and childhood obesity researchers are reporting more frequent cases of obesity-related diseases such as type 2 diabetes, asthma and hypertension that once were considered adult conditions very rare in the past.

There are three centers in the human brain that regulate food intake, the hunger center located in the ventrolateral nucleus of the hypothalamus, the appetite center located in the brain stem and the satiety center in the ventromedial hypothalamus. The hunger and appetite center stimulate an individual to eat, while the satiety center extinguishes the need for food.

Many details of this complete regulatory system are not yet understood. However, it is known that food intake is regulated by both chemical and sensory signals between the brain and the rest of the body. Mouth, stomach, intestines, and nutrient concentration are factors that contribute to appetite and food intake.

The hunger and satiety mechanisms are modulated by sensations elicited by sensory nerve endings located close to the lumen of the gastro intestinal tract and by neurological mechanisms triggered by noxious stimuli.

Gastric and intestinal nerves respond to various types of stimuli including pH, chemical composition of luminal contents or distortion of the mucosa. While we eat and during food digestion our sensory systems are continuously sensing changes in the chemical and physical environment in the GI tract and the information of these changes activate the hypothalamic and brain stem centers.

Our company has recently focused on fine tuning modulatory mechanisms that control food intake and fat burning. We have explored manipulation of visceral sensitivity and thermoregulation rather than hormonal or other more traditional weight loss approaches mitigating hunger by promoting CNS or sympathetic stimulation.

We identified novel weight control treatments using a number of natural sources of essential oils containing high concentration of compounds belonging to the vanilloid class. These chemicals have a unique specificity to bind sensory gastric fibers normally relating information about stomach status to the various brain centers.

Our company has found that vanilloids have two unexpected an unobvious effects that serve to reduce weight, one, is shifting the peripheral thermoregulatory balance to enhanced thermogenesis, resulting in an increase in fat catabolism (oxidation) in the periphery, followed by heat dissipation, and, a second effect, directly on brain cells regulating food intake, body weight and thermoregulation with no central nervous system stimulation. The mechanisms that are four more cases of children who needed help to breath at night because they were so overweight they were chocking on their own fat.

Although the rate of mortality caused by children and adolescents is still low, children and adolescent obesity has many health and social consequences that often continue into adulthood. Pediatricians and childhood obesity researchers are reporting more frequent cases of obesity-related diseases such as type 2 diabetes, asthma and hypertension that once were considered adult conditions very rare in the past.

There are three centers in the mammal brain that regulate food intake, the hunger center located in the ventrolateral nucleus of the hypothalamus, the appetite center located in the brain stem and the satiety center in the ventromedial hypothalamus. The hunger and appetite center stimulate an individual to eat, while the satiety center extinguishes the need for food.

Many details of this complete regulatory system are not yet understood. However, it is known that food intake is regulated by both chemical and sensory signals between the brain and the rest of the body. Mouth, stomach, intestines, and nutrient concentration are factors that contribute to appetite and food intake.

The hunger and satiety mechanisms are modulated by sensations elicited by sensory nerve endings located close to the lumen of the gastro intestinal tract and by neurological mechanisms triggered by noxious stimuli.

Gastric and intestinal nerves respond to various types of stimuli including pH, chemical composition of luminal contents or distortion of the mucosa. While we eat and during food digestion our sensory systems are continuously sensing changes in the chemical and physical environment in the GI tract and the information of these changes activate the hypothalamic and brain stem centers.

Our company has recently focused on fine tuning modulatory mechanisms that control food intake and fat burning. We have explored manipulation of visceral sensitivity and thermoregulation rather than hormonal or other more traditional weight loss approaches mitigating hunger by promoting CNS or sympathetic stimulation.

We identified novel weight control treatments using a number of natural sources of essential oils containing high concentration of compounds belonging to the vanilloid class. These chemicals have a unique specificity to bind sensory gastric fibers normally relating information about stomach status to the various brain centers.

Our company has found that vanilloids have two unexpected an unobvious effects that serve to reduce weight, one, is shifting the peripheral thermoregulatory balance to enhanced thermogenesis, resulting in an increase in fat catabolism (oxidation) in the periphery, followed by heat dissipation, and, a second effect, directly on brain cells regulating food intake, body weight and thermoregulation with no central nervous system stimulation. The mechanisms that are involved in these events are still in progress of being understood but the benefits are therapeutically relevant nonetheless.

The present invention relates to the use of several natural sources of essential oils including cinnamon leaf oil, ginger oil and turmeric oil rich in phenyl alkenes, gingerol and curcumin respectively. These chemicals are present in plants of the family Lauraceae and Zingiberaceae. Most remarkable are Cinnamomum verum J. Presl. (cinnamon), Zingiber officinalis Roscoe (ginger) and Curcuma dornestica Val (turmeric) abundantly consumed in the human diet, especially in China, India and Pakistan, where human adult populations are traditionally slim.

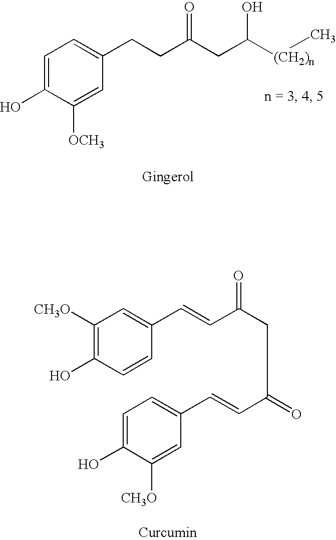

Cinnamon is a small tree originally from India and introduced in the islands of the Indian Ocean and in Southeast Asia and currently cultivated mainly in Sri Lanka. It has been used since 4,000 years for its medicinal and culinary properties. Cinnamon has been reported as a stomachic and carminative for mild gastrointestinal spasms.

Ginger has been used for over 25 centuries in the formulation of countless traditional Chinese remedies, for gastrointestinal complaints and as a remedy against nausea. Multiple clinical trials show the safety of ginger and turmeric in humans.

Turmeric has been used as a yellow food coloring, spice and medicine dating nearly 4,000 years, to the Vedic culture in India, when turmeric was the principal spice. In 1280, Marco Polo described Turmeric as “a vegetable with the properties of saffron, yet it is not really saffron.” Turmeric has been used internally as a medicine in Asia for conditions including headaches, stomach and liver ailments. Turmeric is also used externally to heal sores and as a cosmetic.

It is clear that none of the reported medicinal effects for cinnamon leaf oil, ginger and turmeric oils have been linked to weight control, fat burning or obesity control. However, our company has found a non obvious connection between the pungent effects of these oils for the gastric and intestinal nerve fibers and their ability to control weight in mammals.

We evaluated these oils in a number of in vitro assays and found they display a distinct structure-activity relationship on the mammal hollow viscera, specifically on the rat fundus and rat vas deferens.

We have been able to correlate IC₅₀'s for eugenol, gingerols and curcuminoids and pungency rating in a human tongue assay. We have also correlated the affinity of these chemicals for nerve fibers and their ability to cause hunger reduction, thermogenesis and weight reduction in experimental animals.

We tested therapeutically doses of said oils on human volunteers for over a period of three-six months and found they reduce food intake in humans causing a gradual and sustained decrease in body weight. Additional observations were done in experimental rats especially when these chemicals were combined with citric acid, citrus oil or citrus extractives.

Said composition was free of side effects. The weight reducing benefits were enhanced when cocoa extract containing proanthocyanidines (PAC) and catechins. It has been hypothesized that PAC and catechin suppress fat intake into fat cells. Cocoa also has the virtue to have a negligible amount of caffeine, much less than decaffeinated coffee, and its main constituent, theobromine, is a xanthine that elicits less pronounced effects on the cardiovascular system.

Theobromine concentration in cocoa extract is 6-8% which provides 34 mg in a dose of 50-100 mg, not enough to cause a perceptible brain stimulant event, but we have found this concentration to be sufficient to promote absorption of other constituents and accelerate cellular metabolism.

Animals treated with this formula exhibit normal behavior or equivocal signs of CNS stimulation as demonstrated when using a multidimensional animal screen. It has been theorized that the combination of theobromine, catechin phenols, and phenylethanolamine, cause an unexpected mood elevating effect which in our case may be reflected by a good animal and patient compliance to the formula.

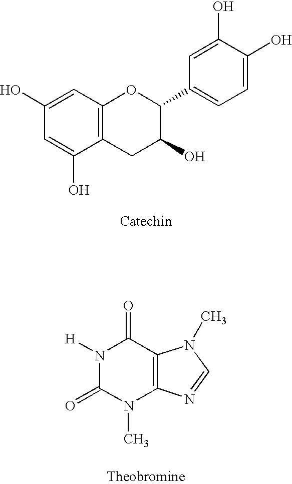

A critical and valuable component of the present invention, is the method of formulation. When you combine essential oils, the vanilloid constituents react among each other and this leads to unwanted changes in the chemical composition, effectiveness and bioavailability of the oils. In order for the formula to be effective it is necessary to apply an unobvious and unexpected method where cinnamon oil, ginger oil and turmeric oil, extracts and oleoresins are individually encapsulated in olive oil an then incorporated in fumed silica.

The encapsulation process entails the individual dissolution of vanilloid-enriched essential oils in olive oil, incorporation of oil-essential droplets into untreated fumed silica, followed by powder mixing following a specific blending methodology to add all ingredients sequentially to produce the final pharmaceutical dosage form.

Said tablet technology is required because of the following unexpected and unobvious situation that leads to the mixing of vanilloid mixtures.
1. It has been found that the essential oils or pure vanilloids present in cinnamon leaf oil, turmeric and ginger may react among each other which lead to the inactivation of the separate constituents.
2. Reactivity of vanilloids is in part due to the presence of a phenolic (acidic) OH group and possibility of molecule addition leading to the loss of a molecule of water.
3. Vanilloids are insoluble in water and require incorporation into an olive oil matrix to facilitate micellar formation and to control delivery and absorption rates.

Vanilloid enriched oils and extracts are individually trapped in a droplet of olive oil embedded in a highly purified and untreated fumed silica matrix. Untreated fume silica has a extremely small particle size, low bulk density and present a highly-branched chain-like structure.

When untreated fumed silica is out in contact with the olive oil it flocks together and form a three-dimensional network of silica and oil droplets. Large voids develop between the silica aggregates. Olive oil fills these void spaces. Consequently, a highly branched structure is formed that provides more efficient thickening and rheology control. The olive oil-essential oil-fumed silica mixture is blended for 3 min at 3,000 RPM which allows the complete integration of the vanilloids in the formulation.

The surface chemistry of untreated fumed silica may be important for this formulation. Fumed silica surface is hydrophilic (water loving) and is capable of hydrogen bonding with the OH groups of the vanilloids. During the formulation of this product, hydroxyl groups of the vanilloids may become attached to some of the silicon atoms on the particle surface. The fumed silica gel containing the droplets of a single essential oil in its interior are then mixed with the other fumed silica gel containing other kind of essential oils. The silica-oil mix is added one by one, so that they don't react with each other and then blended with methyl cellulose and tablet pressed.

The tablet formulation of cinnamon oil, ginger oil and turmeric oil or oleoresins can be improved by the use of citric acid, a good natural preservative which adds an acidic (sour) taste to foods and helps to preserve the composition and prevent fat storage.

Tablets can also be improved by a combination of alpha amylase inhibitors that block the breakdown of starch, thereby reducing free sugar intake which translates in a reduced caloric intake from carbohydrates and less fat accumulation in fat cells. Alpha amylase inhibitors include protein fractions from kidney bean, wheat and potato, to name a few. The amount of alpha amylase provides the opportunity to create 500 mg or 1,000 mg tablets compliant for adults and adolescents.

This tablet remains extremely hygroscopic which allows the fast dissolution of the tablet and liberation of the content in the gastric medium. Additional carriers for this formula include methyl cellulose, crystalline cellulose 101 and crystalline cellulose 102 as well as propylmethyl cellulose.

An additional formulation part of this invention is the method of preparation of a syrup to be used especially by children. The syrup formulation includes curcumin, cinnamon leaf oil and cocoa extract in a distilled water base with sorbitol as a preservative and natural color and flavor additives.

In summary, the present invention is directed to method of formulation of essential oils and pharmaceutical compositions of cinnamon leaf oil, ginger oil and turmeric oil. Said compositions may be formulated as a fast acting or slow release tablets and a syrup, said formulations are effective to control weight and enhance fat metabolism in adults, children and adolescents.

The compositions elicit a reduction of visceral sensitivity, which translates in portion control and a reduction in caloric intake. Additional weight loss is achieved by lipolysis and gluconeogenesis, reduction of fat uptake and storage and carbohydrate blockade by the combination of catechins and proanthocyanidins in other ingredients such as cocoa, citric acid and other constituents in the formula.

It is a primary object of the present invention to provide a formulation method in which cinnamon leaf oil, gingerol and curcumin when combined with acceptable pharmaceutical grade carriers reduce interaction of the phenyl groups and are presented in tablet and syrup forms as a natural dietary supplement to be taken before meals. These supplements elicit heat generation, enhanced fat metabolism and oxidation followed by heat dissipation allowing controlling weight of adults, adolescents and children.

### EXAMPLES

A pharmaceutical composition of cinnamon leaf oil, ginger oil with a high concentration of gingerols, turmeric extract with a high concentration of curcumin are mixed with olive oil and said ingredients are prepared in a tablet combined with either of all of the following: citric acid, citric oil, cocoa extractives and a protein fraction from wheat or kidney bean with alpha amylase inhibitors as described below. Following are examples of tablet formulations of the product.

Tablets

Slow Acting Formulation with High Concentration of Cinnamon Leaf Oil

| Ingredients | Amount per tablet (1-1.5 g) | |
|---|---|---|
| Cinnamon leaf oil (90% eugenol) | 10-20 | mg |
| Ginger oil (20% gingerols) | 1-7 | mg |
| Curcuma oil (95% curcumin) | 0.5-3 | mg |
| Olive oil | 3-10 | mg |
| Cocoa extract (6% Theobromine) | 50-100 | mg |
| Citric acid or citrus oil | 1-5 | mg |
| Protein Fraction from kidney bean or wheat | 500-1000 | mg |
| Fumed Silica | 25-50 | mg |
| Avicel PH 102 | 100-200 | mg |
| Avicel PH 101 | 150-350 | mg |
| Methocel | 100-200 | mg |

Fast Acting Formulation with High Concentration of Cinnamon Leaf Oil

| Ingredient | Amount per tablet (1-1.5 g) | |
|---|---|---|
| Cinnamon leaf oil (90% eugenol) | 10-20 | mg |
| Ginger oil (30% gingerols) | 1-7 | mg |
| Curcuma oil (95% curcumin) | 0.5-3 | mg |
| Olive oil | 3-10 | mg |
| Cocoa extract (6% Theobromine) | 50-100 | mg |
| Citric acid or citrus oil | 1-5 | mg |
| Protein Fraction from kidney bean or wheat | 500-1000 | mg |
| Avicel PH 102 | 150-250 | mg |
| Avicel PH 101 | 400-600 | mg |
| Fumed Silica | 25-50 | mg |

Slow Acting Formulation with Low Concentration of Cinnamon Leaf Oil

| Ingredient | Amount per tablet (1-1.5 g) | |
|---|---|---|
| Ginger oil (30% gingerols) | 10-20 | mg |
| Curcuma oil (95% curcumin) | 1-7 | mg |
| Cinnamon leaf oil (90% eugenol) | 5-10 | mg |
| Olive oil | 3-10 | mg |
| Cocoa extract (6% Theobromine) | 50-100 | mg |
| Citric acid or citrus oil | 1-5 | mg |
| Protein fraction from kidney bean or wheat | 500-1000 | mg |
| Fumed silica | 25-50 | mg |
| Avicel PH 102 | 100-200 | mg |
| Avicel PH 101 | 150-350 | mg |
| Fumed silica | 100-200 | mg |

Fast Acting Formulation with Low Concentration of Cinnamon Leaf Oil

| Ingredient | Amount per tablet (1-2.0 g) | |
|---|---|---|
| Ginger oil (30% gingerols) | 10-20 | mg |
| Curcuma oil (95% curcumin) | 1-7 | mg |
| Cinnamon leaf oil (90% eugenol) | 5-10 | mg |
| Olive oil | 3-10 | mg |
| Cocoa extract (6% Theobromine) | 50-100 | mg |
| Citric acid or citrus oil | 1-5 | mg |
| Alpha amylase from kidney bean | 500-1000 | mg |
| Avicel PH 102 | 150-250 | mg |
| Avicel PH 101 | 400-600 | mg |
| Fumed Silica | 25-50 | mg |

Fast Acting Formulation for Adolescents with Low Concentration of Cinnamon Leaf Oil Smaller Tablets

| Ingredient | Amount per tablet (1-2.0 g) | |
|---|---|---|
| Ginger oil (30% gingerols) | 5-17 | mg |
| Curcuma oil (95% curcumin) | 1-7 | mg |
| Cinnamon leaf oil (90% eugenol) | 2-5 | mg |
| Olive oil | 3-10 | mg |
| Cocoa powder | 50-100 | mg |
| Alpha amylase from kidney bean | 300-800 | mg |
| Avicel PH 102 | 100-250 | mg |
| Avicel PH 101 | 350-600 | mg |
| Fumed Silica | 20-50 | mg |

A pharmaceutical composition in syrup form for children comprises therapeutically effective amounts of cinnamon leaf oil, turmeric extract oil or oleoresin and combinations of these oils with cocoa powder, citric acid, citrus essential oils and a white kidney bean protein fraction with alpha amylase inhibitory activity.

Syrup

Syrup formulation for children with low concentration of cinnamon leaf oil and without ginger to avoid chemical reaction.

| Ingredient | Percent in syrup |
|---|---|
| Curcuma powder (95% curcumin) | 2.0-7.0 |
| Cinnamon leaf oil (90% eugenol) | 2.0-5.0 |
| Olive oil | 0.2-1.0 |
| Cocoa powder | 1.0-10.0 |
| Alpha amylase from kidney bean | 5.0-25.0 |
| Cherry or grape flavor | 0.5-1.0 |
| Natural color | 0.2-0.5 |
| Sorbitol | 6.0-10.0 |
| Distilled Water | 40.0-70.0 |

1. I claim that application of formulation as mentioned on original application regarding adults, adolescents and children (page 8) is also applicable to weight loss control.

2. I claim that the technology to manufacture tablet to combine two or more essential oils in one product avoids adverse chemical reaction.

3. I claim that the above mentioned is water activated. That is, a tablet taken with water will have full effect described in application.
